(19) United States
(12) Patent Application Publication
(10) Pub. No.: US 2011/0092997 A1
(43) Pub. Date: Apr. 21, 2011

(54) MICRO-COIL ASSEMBLY

(76) Inventor: Ho Chang KANG, Seongnam-si (KR)

(21) Appl. No.: 12/627,496

(22) Filed: Nov. 30, 2009

(30) Foreign Application Priority Data
Oct. 16, 2009 (KR) 10-2009-0098928

(51) Int. Cl. A61M 29/00

(52) U.S. Cl. 606/191

(57) ABSTRACT

Disclosed is a micro-coil assembly including: a micro-coil unit which is inserted into an cerebral aneurysm region of a patient and prevents inflow of blood by leading the blood to clot; a coil pusher unit which is arranged adjacent to the micro-coil unit and carries the micro-coil unit to the cerebral aneurysm region of the patient; a tie which connects an end part of the micro-coil unit and the coil pusher unit; and a tensile wire which is relatively movably arranged in the coil pusher unit and coupled to the tie to tense and cut the tie when the micro-coil assembly is separated. Thus, the micro-coil assembly has a simple structure and makes a micro-coil unit and a coil-pusher unit be conveniently and accurately separated, so that the micro-coil unit can be precisely inserted in an cerebral aneurysm region, thereby efficiently meeting a surgical operation of an operator.

### CROSS-REFERENCE TO RELATED APPLICATIONS

This application claims the benefit of Korean Patent Application No. 10-2009-0098928, filed on Oct. 16, 2009, in the Korean Intellectual Property Office, the disclosure of which is incorporated herein in its entirety by reference.

### BACKGROUND

The inventive concept relates to a micro-coil assembly, and more particularly, to a micro-coil assembly which has a simple structure and makes a micro-coil unit and a coil-pusher unit be conveniently and accurately separated, so that the micro-coil unit can be precisely inserted in an cerebral aneurysm region, thereby efficiently meeting a surgical operation of an operator.

A cerebral aneurysm (i.e., acute subarachnoid hemorrhage) refers to cerebrovascular swelling on the wall of an artery because of congenitally weak cerebral artery or because of arteriosclerosis, bacterial infections, a head wound, brain syphilis, etc. Such a cerebral aneurysm is suddenly developed without an initial symptom, and brings extreme pain during an attack of the cerebral aneurysm. 15% of cases die suddenly, 15% die under medical treatment, and 30% survive after treatment but feel the acute aftereffect. Therefore, the cerebral aneurysm may be a very deadly disease.

A cure for the cerebral aneurysm is divided into an invasive therapy and a non-invasive therapy. Of these, the non-invasive therapy fills the cerebral aneurysm with the micro-coil and clots blood, thereby preventing an additional inflow of blood and decreasing risk of a ruptured aneurysm (embolization). The non-invasive therapy has been being widely researched and developed since it can ease the aftereffect due to brain surgery, have advantage of short hospitalization, and so on.

The micro-coil assembly used in the non-invasive therapy roughly includes a micro-coil unit and a coil-pusher unit for carrying the micro-coil unit to an cerebral aneurysm region of a patient. When a front end of the micro-coil unit starts being inserted in the cerebral aneurysm region, an operator separates the micro-coil unit from the coil-pusher unit. As a method of separating the micro-coil unit from the coil-pusher unit, there are a mechanical method, a chemical method, a thermal method, etc.

Among them, the most convenient and accurate method is the mechanical method. A conventional mechanical method for the separation is achieved by releasing a locking state between a hook provided in an end part of the micro-coil unit and a hook provided in an end part of the coil-pusher unit. However, such a releasing method is not only complicated but also difficult to separate the micro-coil unit from the coil-pusher accurately at a desired position and desired timing.

Accordingly, research and development have to be carried out on a micro-coil assembly in which the micro-coil unit can be conveniently and accurately separated from the coil-pusher unit.

### SUMMARY

The present inventive concept is to provide a micro-coil assembly which has a simple structure and makes a micro-coil unit and a coil-pusher unit be conveniently and accurately separated, so that the micro-coil unit can be precisely inserted in an cerebral aneurysm region, thereby efficiently meeting a surgical operation of an operator.

According to an aspect of the present inventive concept, there is provided a micro-coil assembly including: a micro-coil unit which is inserted into an cerebral aneurysm region of a patient and prevents inflow of blood by leading the blood to clot; a coil pusher unit which is arranged adjacent to the micro-coil unit and carries the micro-coil unit to the cerebral aneurysm region of the patient; a tie which connects an end part of the micro-coil unit and the coil pusher unit; and a tensile wire which is relatively movably arranged in the coil pusher unit and coupled to the tie to tense and cut the tie when the micro-coil assembly is separated.

The coil pusher unit may be internally formed with an accommodating space in which the tensile wire can be accommodated and relatively move, and be formed with a first through hole at a previously determined region where the accommodating space and an outside communicate with each other.

The coil pusher unit may include a pusher tube having a tube shape; and a pusher cap formed with the first through hole and coupled to the pusher tube at a side of the pusher tube facing the micro-coil unit, and the tie may tie up the micro-coil unit, the tensile wire and the pusher cap while passing through the first through hole at least once.

The pusher cap may be formed with a second through hole on a lateral wall thereof facing the micro-coil unit, through which the tie passes, and the pusher cap is provided with a stopper that restricts the micro-coil unit from moving toward the pusher cap when the tie is tensed by the tensile wire.

The tie may include a suture.

The first through hole may be provided as a slot formed in the previously determined region of the pusher tube.

The pusher tube may be formed with a spiral pattern spirally formed to be easily bent at one end part thereof facing the pusher cap.

The pusher tube and the pusher cap may be formed as a single body.

The pusher tube and the pusher cap may be separately manufactured with different materials, respectively, and then coupled to each other.

One end part of the tensile wire adjacent to the micro-coil unit may be provided to have a loop shape, and the tie may be connected to the micro-coil unit as passing through the first through hole after being tied to the tensile wire.

The micro-coil unit may include a thrombus-leading coil which is inserted in the cerebral aneurysm region of the patient and transformed into a previously determined shape to clot blood; and an expansion-resistive core which is arranged passing through an inside of the thrombus-leading coil.

One end part of the expansion-resistive core adjacent to the pusher cap may be provided to have a loop shape, a lateral wall of the pusher cap facing the micro-coil unit may be formed with a second through hole through which the tie passes, the other end part of the expansion-resistive core opposite to the one end part may have a ball shape or a shape formed by cutting away from a part of a ball to protect an artery into which the micro-coil unit is inserted from being injured, and the tie may be tied to the tensile wire, pass through the first through hole, an inside of a loop of the expansion-resistive core, and the second through hole, and be tied to the tensile wire.

The expansion-resistive core may be shaped like double loops each of which has a loop shape and a plurality of which are spaced apart from each other in a vertical direction.

The thrombus-leading coil and the expansion-resistive coil may be thermally treated to have a previously determined three-dimensional complex shape or a previously determined two-dimensional shape.

The thrombus-leading coil may include platinum, and the expansion-resistive core may include a polymer.

An outer surface of the thrombus-leading coil may include a thrombus-leading coil protective film made of a polymer material.

An outer surface of the expansion-resistive core may include an expansion-resistive coil protective film for enhancing biocompatibility of the expansion-resistive core and preventing the expansion-resistive core from a chemical change.

### BRIEF DESCRIPTION OF THE DRAWINGS

Exemplary embodiments of the inventive concept will be more clearly understood from the following detailed description taken in conjunction with the accompanying drawings in which:

FIG. 1 is an assembled perspective view of a micro-coil assembly according to an exemplary embodiment of the present inventive concept;

FIG. 2 is an enlarged perspective view of an ‘A’ part in FIG. 1;

FIG. 3 is a perspective view showing a pusher cap of the micro-coil assembly in FIG. 1;

FIG. 4 is a perspective view showing a tensile wire of the micro-coil assembly in

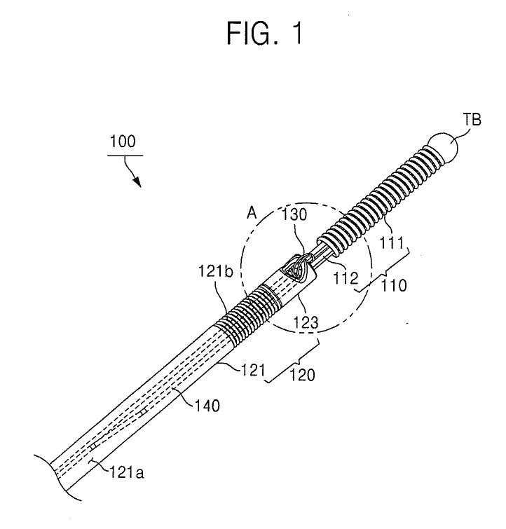

FIG. 1;

FIG. 5 is a perspective view showing that a micro-coil is separated from the micro-coil assembly in FIG. 1;

FIG. 6 is a partial perspective view showing a pusher tube and a protective polymer tube of the micro-coil assembly according to another exemplary embodiment of the present inventive concept;

FIG. 7 is a schematic view showing that the micro-coil assembly of FIG. 1 is inserted in an cerebral aneurysm region of a patient;

FIG. 8 is a schematic view showing that the micro-coil assembly is changed to have a three-dimensional complex shape in the cerebral aneurysm region;

FIG. 9 is a schematic view showing that the micro-coil assembly is changed to have a two-dimensional spiral shape in the cerebral aneurysm region; and

FIG. 10 is a schematic view showing a principle that the micro-coil assembly of FIG. 1 cures the cerebral aneurysm.

### DETAILED DESCRIPTION OF THE EMBODIMENTS

The attached drawings for illustrating embodiments of the inventive concept are referred to in order to gain a sufficient understanding of the inventive concept and the merits thereof.

Hereinafter, the inventive concept will be described in detail by explaining embodiments of the inventive concept with reference to the attached drawings.

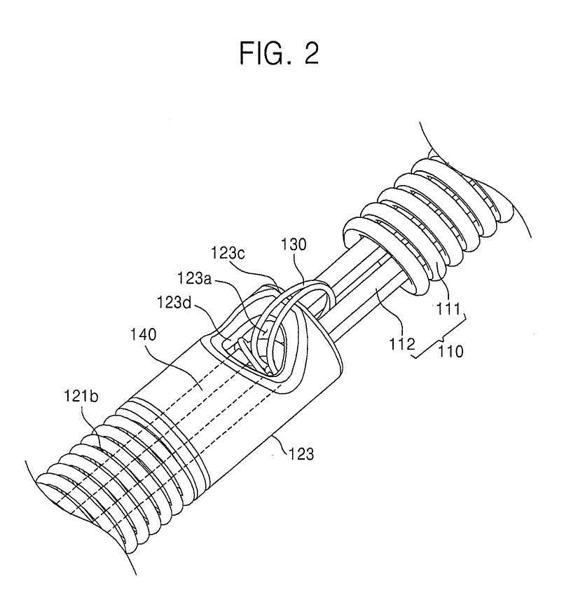

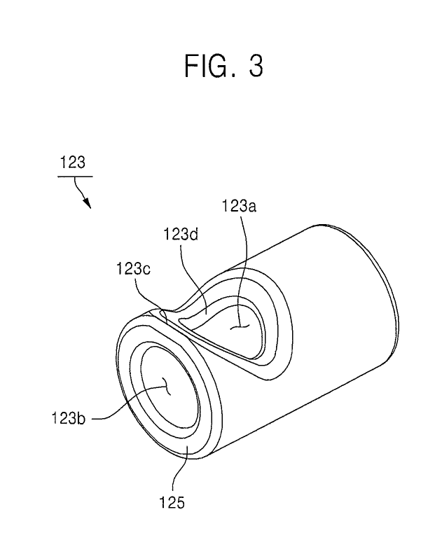

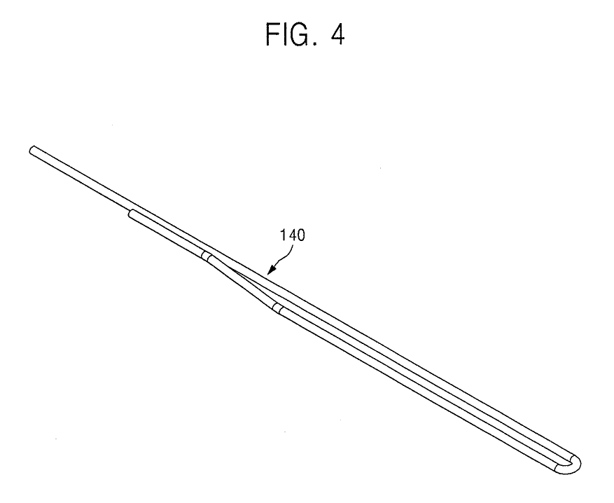

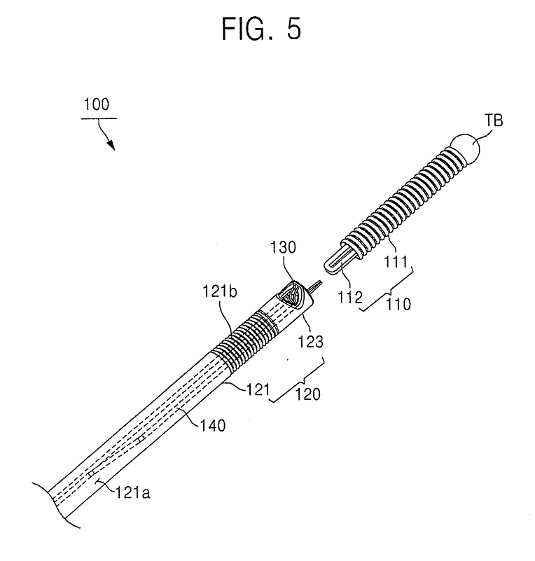

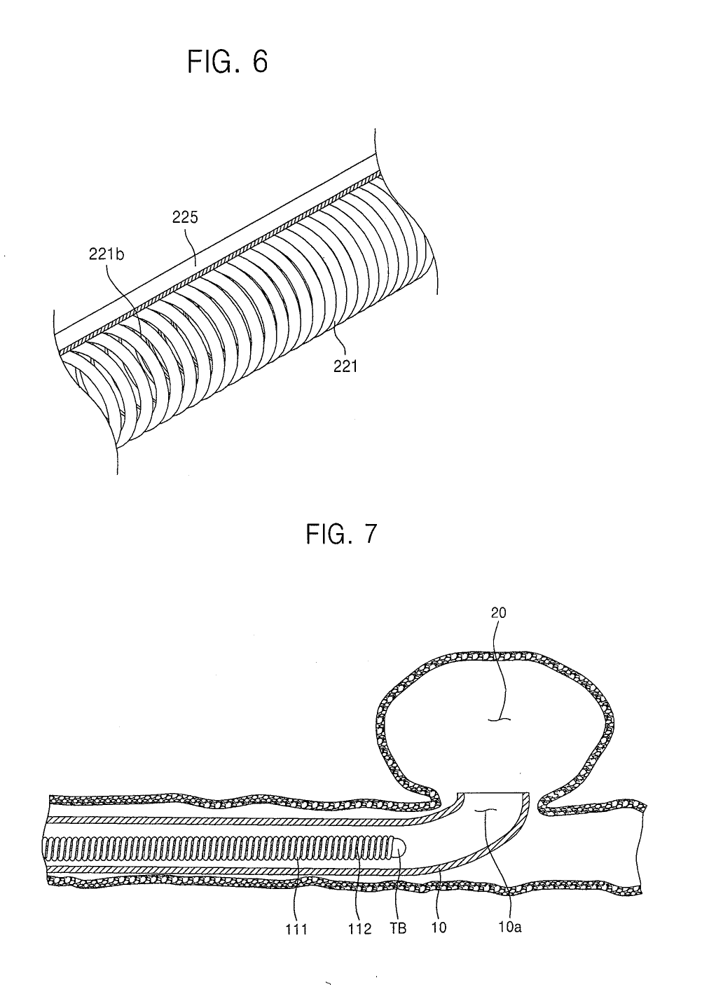

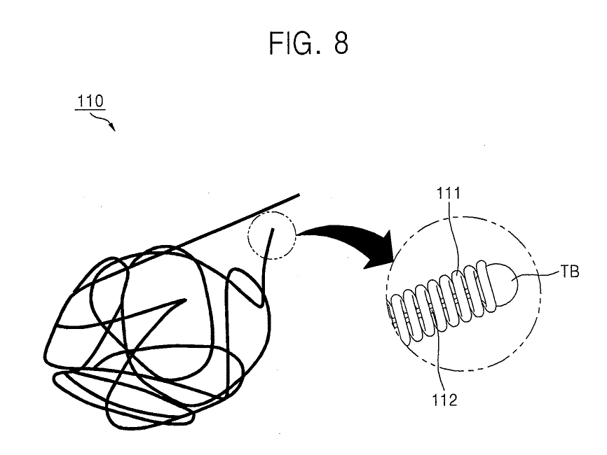

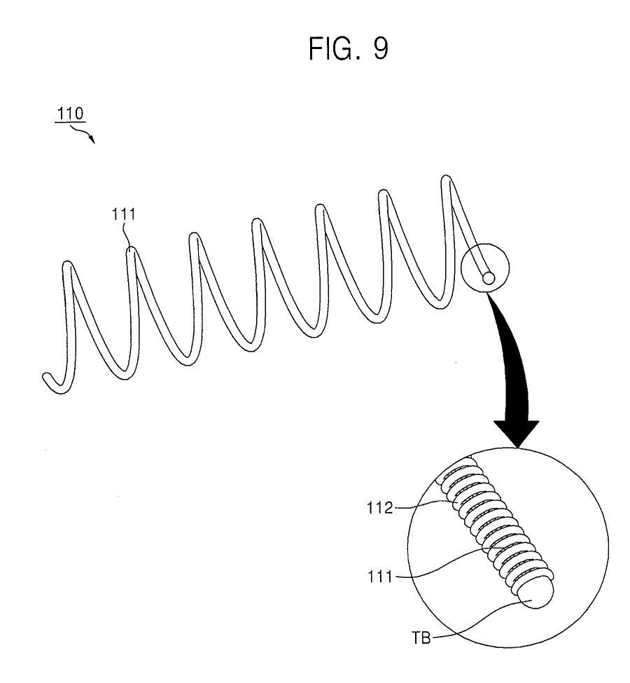

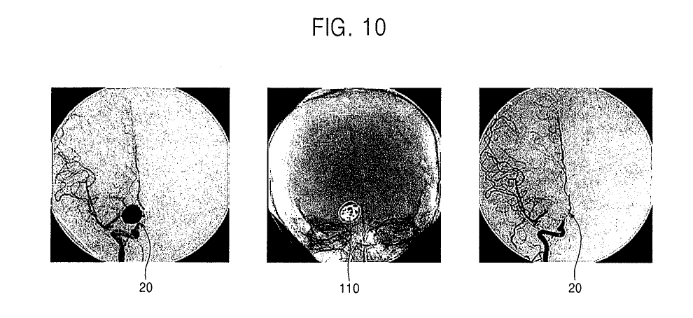

FIG. 1 is an assembled perspective view of a micro-coil assembly according to an exemplary embodiment of the present inventive concept, FIG. 2 is an enlarged perspective view of an ‘A’ part in FIG. 1, FIG. 3 is a perspective view showing a pusher cap of the micro-coil assembly in FIG. 1, FIG. 4 is a perspective view showing a tensile wire of the micro-coil assembly in FIG. 1, and FIG. 5 is a perspective view showing that a micro-coil is separated from the micro-coil assembly in FIG. 1.

As shown therein, a micro-coil assembly 100 in this embodiment includes a micro-coil unit to be inserted in an cerebral aneurysm region of a patient, a coil pusher unit 120 arranged adjacent to the micro-coil unit 110 and carrying the micro-coil unit 110 to the cerebral aneurysm region of the patient, a tie 130 connecting an end part of the micro-coil unit 110 and the coil pusher unit 120, and a tensile wire 140 coupled to the tie 130 and tensing the tie 130 so as to cut the tie 130 when the micro-coil unit 110 is separated. In this embodiment, a suture is used as the tie 130.

The micro-coil unit 110 is inserted into the cerebral aneurysm region of the patient and leads blood to clot, thereby preventing inflow of blood. The micro-coil unit 110 includes a thrombus-leading coil 111 changed to have a previously determined shape and leading blood to clot when inserted in the cerebral aneurysm region of the patient, and an expansion-resistive core 112 penetrating the inside of the thrombus-leading coil 111.

The thrombus-leading coil 111 is manufactured by winding a platinum wire having a proper diameter around a coil-winding device (mandrel) and then applying heat treatment to it in a high-temperature oven. Here, the coil-winding device is provided to have a shape corresponding to the shape of the thrombus-leading coil 111 to be transformed in the cerebral aneurysm of a patient. Also, the proper diameter is determined on the basis of the size of a patient's cerebral aneurysm region, but not limited thereto. Alternatively, the diameter of the thrombus-leading coil 111 may be changed on the basis of the shape of the thrombus-leading coil 111 before the transformation, the flexibility of the thrombus-leading coil 111, the shape of the thrombus-leading coil 111 transformed within the cerebral aneurysm region, etc.

The outer surface of the thrombus-leading coil 111 is coated with a thrombus-leading coil protective film (not shown) made of a polymer. The thrombus-leading coil protective film prevents the thrombus-leading coil 111 from corrosion and assists smooth insertion of the thrombus-leading coil 111 by providing a slippery surface when the thrombus-leading coil 111 is inserted through a micro-catheter. Further, the thrombus-leading coil protective film allows the diameter of the thrombus-leading coil 111 to be reduced so that the thrombus-leading coil 111 can be flexibly designed corresponding to the shape or the size of the cerebral aneurysm region.

The polymer employed as a material for the thrombus-leading coil protective film includes one selected among a fluorohydrocarbon polymer such as tetrafluoroethylene; a hydrophilic polymer such as polyvinylpyrrolidone, polyethyleneoxide or polyhydroxyethylmethacrylate; a polyolefin such as polyethylene or polypropylene; and a polymer such as polyurethane polymer. However, the material of the thrombus-leading coil protective film is not limited thereto, and may be selected from other polymers having a property of matter similar to those of the foregoing polymers.

The expansion-resistive core 112 is changed to have a previously determined shape within the cerebral aneurysm region of the patient, so that the thrombus-leading coil 111 can be accurately positioned within the cerebral aneurysm region. If the thrombus-leading coil 111 is directly pushed or pulled instead of the expansion-resistive core 112, there may be a gap or close contact between the N^{th} winding part and the (N+1)^{th} winding part of the thrombus-leading coil 111 since it is wound spirally.

Accordingly, the expansion-resistive core 112 is provided to solve this problem. An operator (e.g., surgeon or the like) who operates on a patient for the cerebral aneurysm precisely pushes and pulls the expansion-resistive core 112, so that the thrombus-leading coil 111 connected to the expansion-resistive core 112 can be minutely adjusted. That is, the expansion-resistive core 112 is not easily transferred even when pushed or pulled, so that an operator can accurately insert the thrombus-leading coil 111 in the cerebral aneurysm region.

The expansion-resistive core 112 is made of a polymer, which is produced by polymerizing molecules, as being the opposite of a monomer. The expansion-resistive core 112 includes one selected among various kinds of polymers such as polypropylene, nylon, polyamide monofilament, and polyamide composite filament. Polypropylene is a thermoplastic resin produced by polymerizing propylene; nylon is the generic term for a synthesized high molecule polyamaide, which refers to a high molecule shaped like a chain connected with —CONH—; the polyamide monofilament is a monofilament provided with polyamide as a polymer having a structure of an aliphatic or aromatic amide backbone; and the polyamide composite filament is a composite filament provided with polyamide.

The expansion-resistive core 112 made of the polymer is not only flexible but also resistive to the expansion, so that it can be advantageously used as a framing coil, a filling coil or a finishing coil. Here, the framing coil is a coil that is first inserted in the cerebral aneurysm region of the patient and provides a frame to be filled with the filling coil; the filling coil is a coil to be filled in the framing coil; and the finishing coil is a coil to be filled in a minute gap of the framing coil not filled with the filling coil.

Alternatively, the expansion-resistive core 112 may be made of Nitinol. The Nitinol is non-magnetic alloy formed by mixing nickel and titanium in approximately the same ratio.

The outer surface of the expansion-resistive core 112 is formed with parylene coating, polymer coating, polymer tubing, or an expansion-resistive core protective film (not shown) due to passivation of the expansion-resistive core 112. Here, the passivation means various methods for coating or tubing the outer surface of the expansion-resistive core 112 in order to prevent a foreign material or the like from infiltrating into the expansion-resistive core 112. The expansion-resistive core protective film enhances biocompatibility of the expansion-resistive core 112, and prevents the expansion-resistive core 112 from a chemical change due to a chemical reaction between the thrombus thrombus-leading coil 111 and the expansion-resistive core 112.

Meanwhile, one end part of the expansion-resistive core 112 adjacent to the coil pusher unit 120 is shaped like a loop, and the other end part thereof is shaped like a ball or a tip-ball (TB) formed by cutting away a part of the ball. Specifically, the expansion-resistive core in this embodiment is shaped like double loops each of which has a loop shape and which are spaced apart from each other in a vertical direction.

Like this, the one end part of the expansion-resistive core 112 is shaped like a loop, so that the tie 130, i.e., the suture 130 in this embodiment can penetrate the inside of the expansion-resistive core 112 and easily tie the expansion-resistive core 112. That is, as described later, the suture 130 is tied to the tensile wire 140, penetrates a first through hole 123a of the coil pusher unit 120, penetrates the loop of the expansion-resistive core 112 and tied to the tensile wire 140, so that the coil pusher unit 120, the expansion-resistive core 112 and the tensile wire 140 can be connected.

Meanwhile, the other end part of the expansion-resistive core 112 is formed with a tip-ball (TB), so that a wall of an artery can be protected from being injured by the thrombus-leading coil 111 while the thrombus-leading coil 111 is inserted into the cerebral aneurysm region of the patient. The tip-ball is formed by arc-welding the other end part opposite to the one end part adjacent to the coil pusher unit 120 of the expansion-resistive core 112. Particularly, the tip-ball in this embodiment is formed by tungsten inert gas welding (TIG)-welding the other end part of the expansion-resistive core 112, in which the TIG-welding is a tungsten inert gas arc welding method that uses a tungsten rod as an electrode and performs welding while melting a wire by arc through similar manipulation to gas welding.

The TIG-welding is proper to form the tip-ball of the expansion-resistive core 112 in this embodiment since no coating material is used, no slag is generated, and precise welding is possible. However, the present inventive concept is not limited to this method of forming the tip-ball. Alternatively, the tip-ball in this embodiment may be formed by applying not the TIG-welding but another welding method to the other end of the expansion-resistive core 112.

The thrombus-leading coil 111 is fixed to the expansion-resistive core 112 as one end part thereof is in contact with the tip-ball (TB), but not limited thereto. Alternatively, the tip-ball (TB) may be provided by applying the arc-welding between one end part of the thrombus-leading coil 111 and one end part of the expansion-resistive core 112. That is, the tip-ball (TB) may be provided by not applying the welding to the other end part of the expansion-resistive core 112 but applying the arc-welding between one end part of the thrombus-leading coil 111 and the other end part of the expansion-resistive core 112.

The coil pusher unit 120 carries the micro-coil unit 110 to the cerebral aneurysm region of the patient. The coil-pusher unit 120 is internally formed with an accommodating space 121a, so that the tensile wire 140 can be accommodated and relatively move in the accommodating space 121a. Thus, since the tensile wire 140 is relatively movable inside the coil pusher unit 120 with respect to the coil pusher unit 120, the tie 130, i.e., the suture 130 in this embodiment can be tensed to be cut.

The coil pusher unit 120 includes a pusher tube 121 having a tub shape, and a pusher cap 123 having the first through hole 123a through which the suture 130 tied to the tensile wire 140 passes for tying the expansion-resistive core 112 and a second through hole 123b through which the suture 130 tying the expansion-resistive core 112 passes to be tied to the tensile wire 140 again. Here, the pusher tube 121 and the pusher cap 123 may be formed as a single body or separately manufactured and then coupled to each other. In the case where the pusher tube 121 and the pusher cap 123 are separately formed, they may be made of the same material or different materials, respectively.

With this configuration, the suture 130 is tied to the loop of the tensile wire 140 inside the pusher cap 123, passes through the first through hole 123a, ties the expansion-resistive coil 112, passes through the second through hole 123b, and is tied to the tensile wire 140 again. When the micro-coil unit 110 is inserted into the cerebral aneurysm region of the patient, the tensile wire 140 is tensed so that the suture is tensed and broken, thereby separating the micro-coil unit 110.

In the meantime, if the expansion-resistive core 112 tied to the suture 130 continues to move toward the pusher cap 123 when the tensile wire 140 is pulled to cut the suture 130, not only the suture 130 is not cut but also the micro-coil unit 110 is inaccurately inserted in the cerebral aneurysm region of the patient. Accordingly, an end part of the pusher cap 123 facing the micro-coil unit 110 is provided with a stopper 125 that restricts the micro-coil unit 110 from moving toward the pusher cap 123 when the tensile wire 140 tenses the suture 130.

With this configuration, if the tensile wire 140 tenses the suture 130, the micro-coil unit 110 is caught in the stopper 125 of the pusher cap 123 and the suture 130 becomes tensed while contacting an edge part 123c of the pusher cap 123 and is finally cut, so that the micro-coil unit 110 can be separated and inserted into the cerebral aneurysm region of the patient. At this time, since the suture 130 is tied to the tensile wire 140, the suture 130 is still maintained as it is tied to the tensile wire 140 even though the suture 130 is cut. Accordingly, the suture 130 can be collected by the tensile wire 140 after separating the micro-coil unit 110.

The pusher tube 121 may be made of metal alloy such as Nitinol or 300-series stainless steel; a rigid polymer such as polyetheretherketon (PEEK); or a rigid polymer tube formed by mechanically combining the rigid polymer and the metal alloy.

Further, one end part of the pusher tube 121 facing the pusher cap 123 is formed with a spiral pattern 121b spirally patterned to be bent, but not limited thereto. Alternatively, a plurality of slots spaced apart from each other may be provided. Further, if the pusher tube 121 is made of a material such as nylon, there may not be provided the spiral pattern 121b or the plurality of slots spaced apart from each other.

Also, as shown in FIG. 6, according to another exemplary embodiment of the present inventive concept, the spiral pattern 221b spirally patterned to be easily bent is formed through approximately the whole area of the pusher tube 221, and a protective polymer tube may be put on the outer surface of the pusher tube 221 formed with the spiral pattern 221b. Here, the spiral pattern 221b is formed approximately throughout the pusher tube 221, so that it can be bent smoothly and prevented from being broken at a certain position. Referring to FIG. 6, if the pitch of the spiral pattern 221b becomes larger as the spiral pattern 221b gets more distant from the pusher cap (not shown), it becomes more flexible as getting closer to the pusher cap (not shown). Also, if the protective polymer tube 225 is put on and coupled to the outer surface of the pusher tube 221 formed with the spiral pattern 221b, the pusher tube 221 is prevented from being flexible in a shaft direction. Accordingly, the spiral pattern 221b does not cause the pusher tube 221 to be flexible in the shaft direction, thereby making the operation easy.

Returning back to the first exemplary embodiment, the pusher cap 123 is made of metal alloy, preferably, platinum or 300-series stainless steel hypotube or radiopaque. On a top part of the pusher cap 123 is formed with a slot 123d forming the first through hole 123a through which the suture 130 passes. If the pusher cap 123 is made of a material different from that of the pusher tube 121, an adhesive or other proper coupling technology are needed to couple them. If the pusher tube 121 and the pusher cap 123 are formed as a single body, they may be made of PEEK having sufficient rigidity.

The suture 130 ties up the expansion-resistive coil 112, the tensile wire 140 and the pusher cap 123. The suture 130 is pulled by the tensile wire 140 when the micro-coil unit 110 is separated, and finally tensed and cut. The suture 130 may be provided in the form of a single loop or multi loops according to requested tensile strength, and may include a monofilament, a multifilament, or the like substances.

The tensile wire 140 pushes or pulls the suture 130. The tensile wire 140 pulls and breaks the suture 130 when the micro-coil unit 110 is separated. The tensile wire 140 is formed by bending one end part thereof to form a loop shape and welding or soldering the end of the one end part to the other part thereof not bent. Preferably, the tensile wire 140 is made of metal alloy having the minimum elasticity or the 300-series stainless steel.

Below, a method of using the micro-coil assembly 100 in this embodiment will be schematically described.

FIG. 7 is a schematic view showing that the micro-coil assembly of FIG. 1 is inserted in an cerebral aneurysm region of a patient, FIG. 8 is a schematic view showing that the micro-coil assembly is changed to have a three-dimensional complex shape in the cerebral aneurysm region, FIG. 9 is a schematic view showing that the micro-coil assembly is changed to have a two-dimensional spiral shape in the cerebral aneurysm region, and FIG. 10 is a schematic view showing a principle that the micro-coil assembly of FIG. 1 cures the cerebral aneurysm.

Referring to FIGS. 7 to 10 together with FIGS. 1 and 5, the micro-coil assembly 100 is inserted into the cerebral aneurysm region 20 on the artery along the inside 10a of the micro-catheter 10 extended from a proper insertion starting position such as the femoral region of the patient to the cerebral aneurysm region. That is, the micro-catheter 10 extended to cerebral aneurysm region 20 is first inserted, and then the micro-coil assembly 100 is inserted along the micro-catheter 10. The micro-coil assembly 100 is manufactured to have a very small diameter and thus have certain flexibility inside the micro-catheter 10, so that it can be conveniently inserted.

The micro-coil unit 110 connected to the coil pusher unit 120 is not randomly deformed within the micro-catheter 10 according to the stress on the inner wall of the micro-catheter 10, and is carried as it is to the cerebral aneurysm region 20.

If the micro-coil unit 110 is inserted into the cerebral aneurysm region 20 of the patient, the suture 130 is pulled through the tensile wire 140. Then, the micro-coil unit 110 is restricted from moving by the stopper 125 of the pusher cap 123, so that the suture 130 is extended between the tensile wire 140 and the pusher cap 123 while the suture 130 is pulled. If the suture 130 reaches the limit of the extension, it is broken. If the suture 130 is broken, it is released from the expansion-resistive core 112 and the pusher cap 123. At this time, since the suture 130 is tied to the tensile wire 140, both ends of the suture 130 is pulled along with the tensile wire 140 even though the suture 130 is broken. Further, as the suture 130 is pulled from the pusher cap 123, the micro-coil unit 110 is completely separated from the pusher cap 123.

It will be noted that tension is not applied to the micro-coil unit 110 while the suture 130 is extended. In other words, the tension is applied only between the pusher cap 123 and the tensile wire 140, so that the micro-coil unit 110 can be free from any load while the suture 130 is cut.

As the suture 130 is broken, the micro-coil unit 110 is separated from the coil pusher unit 120 and completely inserted in the cerebral aneurysm region 20.

The micro-coil unit 110, which comes out of the end of the micro-catheter 10 and is inserted in the cerebral aneurysm region 20, is released from the stress applied by the inner wall of the micro-catheter 10, so that it can be transformed to have a previously determined shape while undergoing the heat treatment, thereby filling the cerebral aneurysm region 20.

As shown in detail in FIGS. 8 and 9, the micro-coil unit 110 comes out of the end of the micro-catheter 10 and is transformed to have a preset random shape such as a two-dimensional spiral shape or a three-dimensional spiral complex pattern. The transformed shape of the micro-coil unit 110 is previously determined depending on the size, the shape and other various data of the cerebral aneurysm region 20 of the patient.

In the micro-coil assembly 100 according to this embodiment, the micro-coil unit 110 and the coil pusher 120 are connected by the suture 130, and the suture 130 is broken by the tensile wire 140, thereby separating the micro-coil unit 110 and the coil pusher 120. Thus, the micro-coil assembly has a simple structure and makes a micro-coil unit and a coil-pusher unit be conveniently and accurately separated, so that the micro-coil unit can be precisely inserted in an cerebral aneurysm region, thereby efficiently meeting a surgical operation of an operator.

In the foregoing embodiment, the tie is the suture, but not limited thereto. Alternatively, various strings or cords may be used as the tie as long as the tensile wire connected to the tie can tense and break the tie.

While the inventive concept has been particularly shown and described with reference to exemplary embodiments thereof, it will be understood that various changes in form and details may be made therein without departing from the spirit and scope of the following claims.

What is claimed is:

1. A micro-coil assembly comprising:
a micro-coil unit which is inserted into an cerebral aneurysm region of a patient and prevents inflow of blood by leading the blood to clot;
a coil pusher unit which is arranged adjacent to the micro-coil unit and carries the micro-coil unit to the cerebral aneurysm region of the patient;
a tie which connects an end part of the micro-coil unit and the coil pusher unit; and
a tensile wire which is relatively movably arranged in the coil pusher unit and coupled to the tie to tense and cut the tie when the micro-coil assembly is separated.

2. The micro-coil assembly according to claim 1, wherein the coil pusher unit is internally formed with an accommodating space in which the tensile wire can be accommodated and relatively move, and is formed with a first through hole at a previously determined region where the accommodating space and an outside communicate with each other.

3. The micro-coil assembly according to claim 2, wherein the coil pusher unit comprises
a pusher tube having a tube shape; and
a pusher cap formed with the first through hole and coupled to the pusher tube at a side of the pusher tube facing the micro-coil unit, and
the tie ties up the micro-coil unit, the tensile wire and the pusher cap while passing through the first through hole at least once.

4. The micro-coil assembly according to claim 3, wherein the pusher cap is formed with a second through hole on a lateral wall thereof facing the micro-coil unit, through which the tie passes, and
the pusher cap is provided with a stopper that restricts the micro-coil unit from moving toward the pusher cap when the tie is tensed by the tensile wire.

5. The micro-coil assembly according to claim 3, wherein the tie comprises a suture.

6. The micro-coil assembly according to claim 3, wherein the first through hole is provided as a slot formed in the previously determined region of the pusher tube.

7. The micro-coil assembly according to claim 3, wherein the pusher tube is formed with a spiral pattern spirally formed to be easily bent at one end part thereof facing the pusher cap.

8. The micro-coil assembly according to claim 3, wherein the pusher tube and the pusher cap are formed as a single body.

9. The micro-coil assembly according to claim 3, wherein the pusher tube and the pusher cap are separately manufactured with different materials, respectively, and then coupled to each other.

10. The micro-coil assembly according to claim 3, wherein one end part of the tensile wire adjacent to the micro-coil unit is provided to have a loop shape, and the tie is connected to the micro-coil unit as passing through the first through hole after being tied to the tensile wire.

11. The micro-coil assembly according to claim 3, wherein the micro-coil unit comprises
a thrombus-leading coil which is inserted in the cerebral aneurysm region of the patient and transformed into a previously determined shape to clot blood; and
an expansion-resistive core which is arranged passing through an inside of the thrombus-leading coil.

12. The micro-coil assembly according to claim 11, wherein
one end part of the expansion-resistive core adjacent to the pusher cap is provided to have a loop shape,
a lateral wall of the pusher cap facing the micro-coil unit is formed with a second through hole through which the tie passes,
the other end part of the expansion-resistive core opposite to the one end part has a ball shape or a shape formed by cutting away from a part of a ball to protect an artery into which the micro-coil unit is inserted from being injured, and
the tie is tied to the tensile wire, passes through the first through hole, an inside of a loop of the expansion-resistive core, and the second through hole, and is tied to the tensile wire.

13. The micro-coil assembly according to claim 12, wherein the expansion-resistive core is shaped like double loops each of which has a loop shape and a plurality of which are spaced apart from each other in a vertical direction.

14. The micro-coil assembly according to claim 11, wherein the thrombus-leading coil and the expansion-resistive coil are thermally treated to have a previously determined three-dimensional complex shape or a previously determined two-dimensional shape.

15. The micro-coil assembly according to claim 11, wherein the thrombus-leading coil comprises platinum, and the expansion-resistive core comprises a polymer.

16. The micro-coil assembly according to claim 11, wherein an outer surface of the thrombus-leading coil comprises a thrombus-leading coil protective film made of a polymer material.

17. The micro-coil assembly according to claim 11, wherein an outer surface of the expansion-resistive core comprises an expansion-resistive coil protective film for enhancing biocompatibility of the expansion-resistive core and preventing the expansion-resistive core from a chemical change.
